(19) United States
(12) Patent Application Publication
(10) Pub. No.: US 2007/0027368 A1
(43) Pub. Date: Feb. 1, 2007

(54) 3D anatomical visualization of physiological signals for online monitoring

(76) Inventors: John Patrick Collins, Cranbury, NJ (US); Mohan Singh, Bayern (DE); Bernd Wachmann, Lawrenceville, NJ (US); Brad Wehrwein, Framingdale, ME (US); Marcela de Castro Esteves, Princeton, NJ (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/481,164

(22) Filed: Jul. 5, 2006

Related U.S. Application Data
(60) Provisional application No. 60/699,419, filed on Jul. 14, 2005.

(51) Int. Cl. A61B 5/00

(52) U.S. Cl. 600/300

(57) ABSTRACT

A method for visualization of a physiological signal, comprising the steps of acquiring a time-series signal from the physiological signal, identifying a patient condition from the time-series signal, displaying a 3D image of a body, and displaying a visual indicator representative of the patient condition on the 3D image of a body.

### CROSS-REFERENCE TO RELATED APPLICATIONS

This application claims priority to U.S. Provisional Application No. 60/699,419, filed on Jul. 14, 2005, the disclosure of which is incorporated by reference herein.

### BACKGROUND OF THE INVENTION

1. Technical Field

The present disclosure relates generally to the field of medical imaging, and, more particularly, to 3D visualization of physiological signals for online monitoring.

2. Discussion of the Related Art

Monitoring physiological signals is commonly realized by the visualization of time series signals from heart rate, respiratory rate, blood oxygen saturation and blood pressure. These data are used to identify critical states to trigger an alarm when the physical condition of a patient becomes critical. The screen of an existing Intensive Care Unit (ICU) monitoring system is shown in FIG. 1.

The users of such monitoring systems (doctors and nurses) need to visually examine time series signals, plotted as values over time, to identify and control the current state of the patient. These signals often contain complex patterns and relationships between various channels that are not quickly identifiable by a human expert, especially when the time series only spans a couple of seconds. Furthermore, with multiple time series signals, it is not immediately evident to the user which signal is related to a specific physiological condition or organ, because the logical connection between the interpretation of the data and the plot of the data is usually expressed only with a textual label.

There exists a need for a method and apparatus for visually representing physiological signals to make the diagnosis of patient conditions more efficient.

### SUMMARY OF THE INVENTION

An exemplary embodiment of the present invention provides a method for anatomical visualization of a physiological signal, comprising the steps of: acquiring a time-series signal from the physiological signal, identifying a patient condition from the time-series signal, displaying a 3D image of a body, and displaying a visual indicator representative of the patient condition on the 3D image of a body. The visual indicator may appear as a 3D anatomical structure representative of the corresponding physiological signal. The 3D anatomical structure may change periodically in color and brightness to indicate that the patient condition is critical or approaching a critical state. An audible alarm may be sounded when the patient condition is exceedingly critical. The 3D image of a body may be derived from computer tomography data of a patient.

An exemplary embodiment of the present invention provides a method for visualizing a plurality of physiological signals, comprising the steps of: acquiring a plurality of time-series signals from the corresponding plurality of physiological signals, identifying a plurality of patient conditions from the time-series signals, displaying a 3D image of a body, and displaying a plurality of visual indicators on the 3D image of a body which correspond to the patient conditions.

An exemplary embodiment of the present invention provides an apparatus for visualization of a plurality of physiological signals, comprising a time-series signal generation unit for acquiring a plurality of physiological signals and generating a plurality of time-series signals, a patient condition analyzer unit for analyzing the time-series signals and generating patient condition data, and a display unit for displaying the patient condition data as a 3D anatomical structure on a 3D template body.

### BRIEF DESCRIPTION OF THE DRAWINGS

The invention may be understood by reference to the following description taken in conjunction with the accompanying drawings, in which like reference numerals identify like elements, and in which:

FIG. 1 illustrates a conventional Intensive Care Unit (ICU) monitoring system.

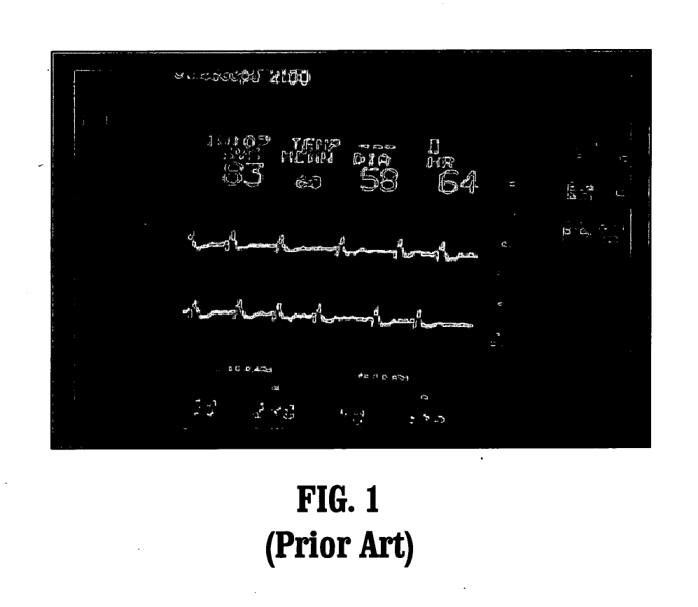

FIG. 2 is a flowchart illustrating a method for anatomical visualization of a physiological signal according to an exemplary embodiment of the present invention.

FIG. 3a and FIG. 3b illustrate a 3D anatomical condition visualization of a respiratory physiological signal according to an exemplary embodiment of the present invention.

FIG. 4 illustrates an apparatus for visualization of a plurality of physiological signals according to an exemplary embodiment of the present invention.

FIG. 5a illustrates a graphical user interface of a monitoring system for an ICU patient according to an exemplary embodiment of the present invention.

FIG. 5b illustrates a Search Event function of the graphical user interface of FIG. 4a.

### DETAILED DESCRIPTION OF EXEMPLARY EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

It is to be understood that the systems and methods described herein may be implemented in various forms of hardware, software, firmware, special purpose processors, or a combination thereof. In particular, at least a portion of the present invention is preferably implemented as an application comprising-program instructions that are tangibly embodied on one or more program storage devices (e.g., hard disk, magnetic floppy disk, RAM, ROM, CD ROM, etc.) and executable by any device or machine comprising suitable architecture, such as a general purpose digital computer having a processor, memory, and input/output interfaces. It is to be further understood that, because some of the constituent system components and process steps depicted in the accompanying Figures are preferably implemented in software, the connections between system modules (or the logic flow of method steps) may differ depending upon the manner in which the present invention is programmed. Given the teachings herein, one of ordinary skill in the related art will be able to contemplate these and similar implementations of the present invention.

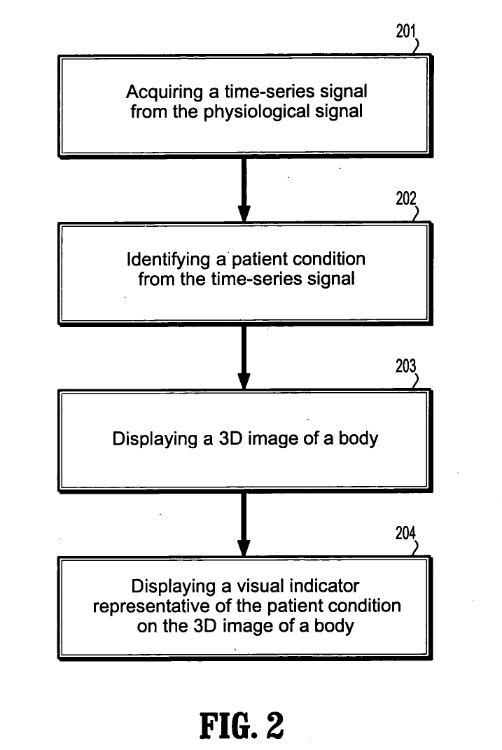

FIG. 2 is a flowchart illustrating a method for anatomical visualization of a physiological signal according to an exemplary embodiment of the present invention.

Referring to FIG. 2, in a step 201, a time series signal is acquired from a physiological signal. A physiological signal may be derived from a human body using a biomedical transducer or any other suitable data gathering tool. The physiological signal may be filtered to suppress noise and normalized. When the physiological signal is plotted over time, a time series signal may be generated.

In a step 202, a patient condition is identified from the time series signal. The methods of identifying the patient condition vary according to the physiological signal because each physiological signal can generate a very different corresponding time series signal. In addition, there are various methods of analyzing a particular time series signal based on the knowledge in the art of biomedical signal analysis. The patient condition may be identified by periodically extracting statistics from the time series signal over a period of time. The statistics can then be compared to a predetermined library of statistical models which each correspond to a particular patient condition. Examples of the statistics include moving averages, min/max values over a predefined time interval, and slope changing information (i.e., the tendency of a signal to move downward/upward), etc. The patient condition may be identified when a statistical model matches the extracted statistics. However, the present invention is not limited to any particular identification method.

In a step 203, a 3D image of a body is displayed. The 3D image of the body may be substantially identical to the actual body of a patient that the physiological signal was derived from, or it may be selected from a set of generic body templates based on the sex and age of the patient.

In a step 204, a visual indicator of the patient condition is displayed on the 3D image of the body. The visual indicator is representative of the physiological condition. The visual indicator may be a 3D image of an organ corresponding to the physiological signal or any other visually indicative graphic.

In an exemplary embodiment of the present invention, there is provided a computer readable medium including computer code for visualizing a plurality of physiological signals, the computer readable medium comprising: computer code for acquiring a time series signal from a physiological signal, computer code for identifying a plurality of patient conditions from the time-series signals, and computer code for displaying a 3D image of a body and displaying a plurality of visual indicators on the 3D image of a body which correspond to the patient conditions.

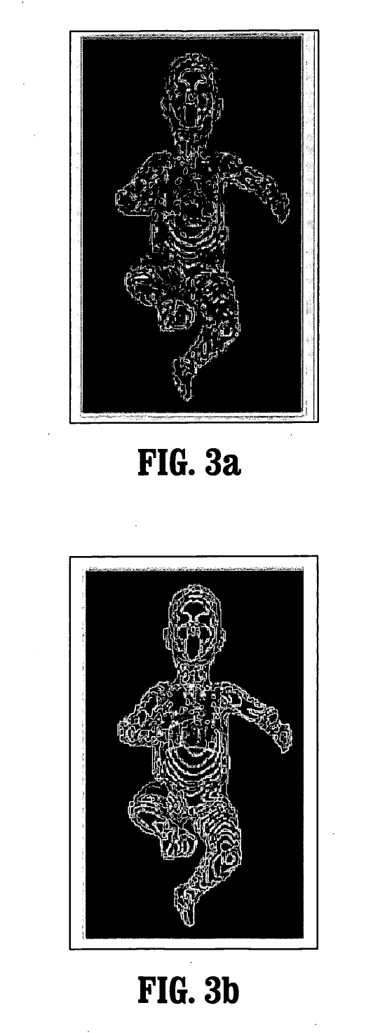

FIG. 3a and FIG. 3b illustrate a 3D anatomical condition visualization of a respiratory rate physiological signal according to an exemplary embodiment of the present invention.

FIG. 3a and 3b make use of a 3D image of a template body and lungs to represent the respiratory rate of an infant patient. Although the template body displayed in these figures is that of an infant, the present invention is not limited to infants, and applies to any patient type including adults and adolescents. The template body may resemble the patient in a general way by making use of generic body templates such as adult female, adult male, adolescent female, adolescent female, infant, etc. However, the body template may also be derived from actual patient computer tomography data to more accurately depict the patient. The 3D image of lungs in FIG. 3a visually illustrate that the respiratory rate of the infant patient is normal or stable. In an exemplary embodiment of the present invention, the 3D image of lungs on the template body is displayed in a color to indicate normal or stable breathing. However, when the respiratory rate falls outside the normal range (i.e., below a critical value or even zero), the image in FIG. 3b is displayed. In an exemplary embodiment of the invention, the 3D image of lungs are displayed in a color to indicate that the infant's breathing is either in a critical state that needs attention, or that the patient's breathing is deteriorating and is expected to reach a critical state.

Any number of colors may be used to indicate both normal and abnormal breathing conditions. The color which indicates abnormal breathing may also blink at a predetermined rate to act as a visual cue to facilitate diagnosis. A blinking color may be produced by alternately displaying a color and a version of the same color at a different intensity or brightness at a periodic rate. The present invention is not limited to use of color changes to indicate abnormal or critical conditions. Texture of the drawn anatomical structure could be used to differentiate a stable condition from an abnormal or critical condition. As an example, the 3D image of lungs could be displayed as transparent when stable and then displayed with a hatched pattern to indicate the abnormal or critical condition. Textual labels could also be used to differentiate between stable and critical conditions. As an example, when the patient is experiencing Apnea (difficulty breathing), a blinking letter A for Apnea could be superimposed over the 3D image of lungs in the template body.

While the 3D image of lungs in FIG. 3a and 3b is representative of a diagnosis of a time-series signal of a respiratory rate physiological signal, the present invention may be applied to various physiological signals including blood pressure, blood oxygen saturation, heart rate, etc. When the respiratory rate is deemed to be exceedingly critical, an audible alarm may be sounded in addition to the anatomical visual indicator comprising the 3D image of lungs.

In an exemplary embodiment of the present invention, when the physiological signal is of blood pressure, a 3D vessel structure is displayed on the template body. When the blood pressure of a patient is stable, the 3D vessel structure is displayed in a color that indicates blood pressure is stable. When the blood pressure is abnormal (i.e., too high or too low), or is expected to become abnormal, the color of the 3D vessel structure changes to a color which indicates that blood pressure is abnormal or likely to become abnormal. The resulting color may blink as described above, acting as a visual cue to the user. Low and high pressure may be indicated by different colors. When the blood pressure is deemed to be exceedingly critical, an audible alarm may be sounded in addition to the anatomical visual indicator comprising the 3D vessel structure. Texture changes to the vessels may be used to differentiate between normal and abnormal blood pressure. As an example, the vessels of the 3D vessel structure may appear hollow when the blood pressure is stable and hatched when blood pressure is either too high or too low. Textual labels may also be used to differentiate between abnormal and normal blood pressure. As a further example, a blinking letter H could be superimposed over the 3D vessel structure to indicate high blood pressure while a blinking letter L could be used to indicate low blood pressure.

In an exemplary embodiment of the present invention, when the physiological signal is of blood oxygen saturation, a 3D image of skin is displayed on the template body. Since diagnosis of various physiological signals may be displayed on the template body, the 3D image of skin may be transparent to prevent obscuration of other anatomical structures. When the blood oxygen saturation of a patient is stable, the 3D image of skin is displayed in a color that indicates stable blood oxygen saturation. When the blood oxygen saturation of a patient is abnormal (i.e., too low or fluctuating too strongly) or is expected to become abnormal, the 3D image of skin changes to a color which indicates blood oxygen saturation is abnormal or likely to become abnormal. The color which indicates a stabile blood oxygen saturation may be exemplified as red. The color which indicates an abnormal blood oxygen saturation may be exemplified as blue and blink as described above to act as a visual cue to the user. Low blood oxygen saturation and oxygen blood saturation that fluctuates too strongly may be indicated by different colors. When the blood oxygen saturation is deemed to be exceedingly critical, an audible alarm may be sounded in addition to the anatomical visual indicator comprising the skin. Texture and textual labels may also be used to differentiate between stable and critical blood oxygen saturation levels.

In an exemplary embodiment of the present invention, when the physiological signal is of heart rate, a 3D image of a heart is displayed on the template body. When the heart rate of a patient is stable, the 3D image of the heart is displayed in a color that indicates a stable heart rate. However, when the heart rate of a patient is abnormal (i.e., too low or too high) or is expected to become abnormal, the 3D image of the heart changes to a color which indicates heart rate is abnormal or likely to become abnormal. The color which indicates an abnormal heart rate may blink as described above to act as a visual cue to the user. Low and high blood heart rates may be indicated by different colors. When the heart rate is deemed to be exceedingly critical, an audible alarm may be sounded in addition to the anatomical visual indicator comprising the heart. Texture may be used to differentiate between stable and critical heart rates. As an example, the 3D image of the heart may appear transparent when the heart rate is stable and with a hatched pattern when the heart rate is critical. Textual labels may also be used to differentiate between stabile and critical heart rates. As a further example, a blinking letter H could be superimposed over the 3D image of the heart to indicate a rapid heart rate, while a blinking L could be superimposed over the 3D image of the heart to indicate a sluggish heart rate.

When multiple physiological signals are being examined through multiple channels, the body template pictured in FIGS. 3a and 3b may simultaneously display all of the anatomical structures described in the exemplary embodiments above, such as lungs for respiratory rate, vessel structure for blood pressure, skin for blood oxygen saturation, and a heart for heart rate. The body template is not limited to displaying diagnosis of blood pressure, blood oxygen saturation, heart rate and respiratory rate. Diagnosis of any number of physiological signals may be represented with varying anatomical structures on the template body. For example, an electroencephalogram (EEG) physiological signal could be represented by a brain.

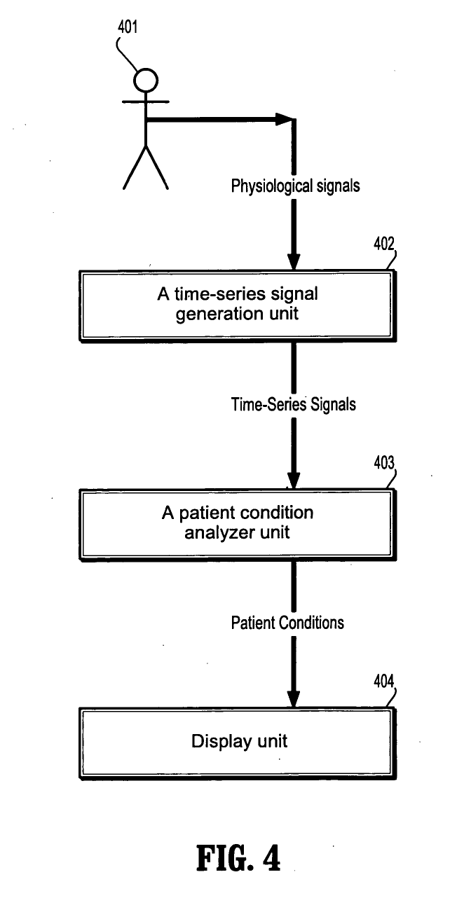

FIG. 4 illustrates an apparatus for visualization of a plurality of physiological signals according to an exemplary embodiment of the present invention.

Referring to FIG. 4, physiological signals are sent from a patient 401 to a time-series signal generation unit 402. Time-series signals are then generated from each of the corresponding physiological signals and then sent to a patient condition analyzer unit 403. Patient condition data is then sent to the display unit 404 and displayed as a 3D anatomical structure on a 3D template body. The 3D anatomical structure may be representative of a corresponding one of the physiological signals. The 3D anatomical structure may change periodically in color and brightness when the patient condition data indicates a critical patient condition. The anatomical structure may remain a constant color when the patient condition data indicates a stable patient condition. The apparatus may further comprise an alarm unit for sounding an audible alarm when the patient condition data indicates an exceedingly critical patient condition.

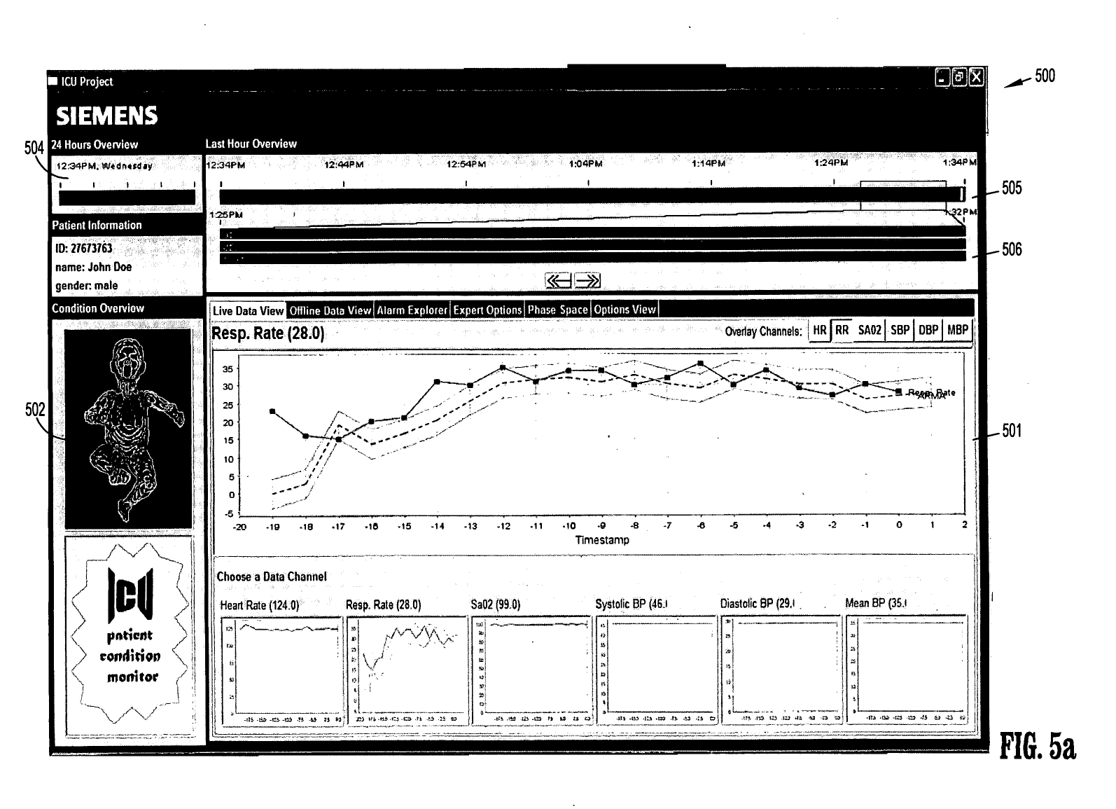

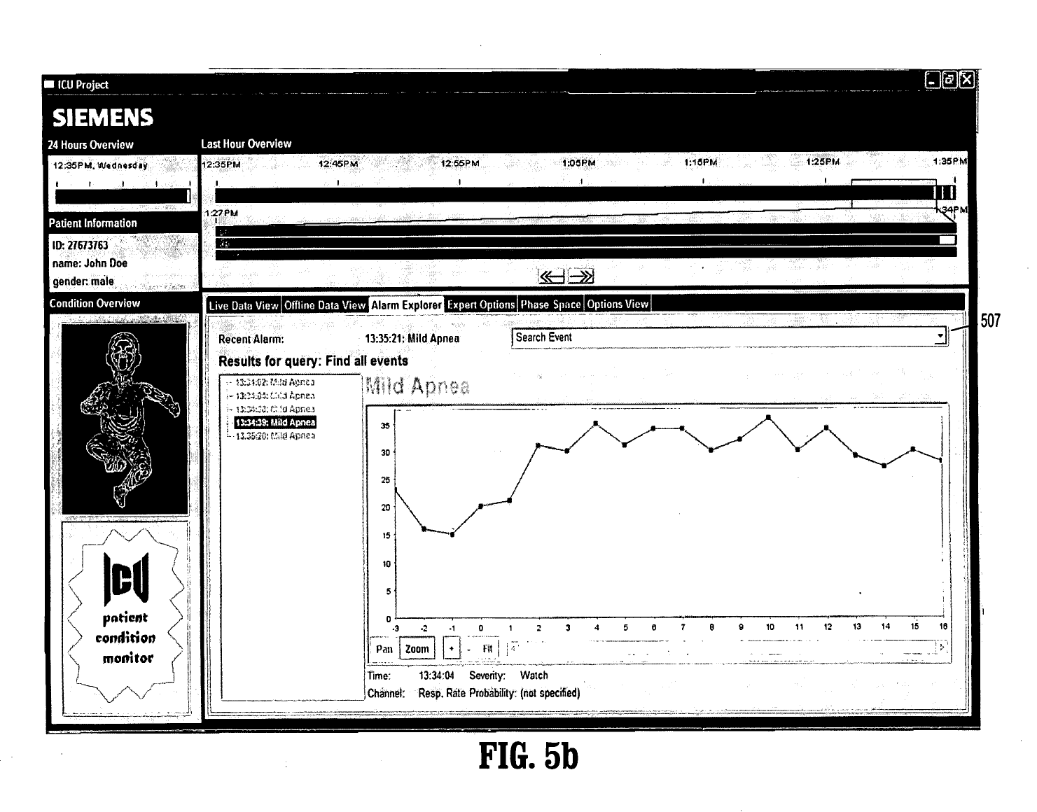

FIG. 5a illustrates a graphical user interface of a monitoring system for an ICU patient according to an exemplary embodiment of the present invention. FIG. 5b illustrates a Search Event function of the graphical user interface of FIG. 5a.

Referring to FIG. 5a, the graphical user interface 500 includes a time series plot section 501 for combined visualization of multiple time series signals, and a 3D anatomical condition visualization section 502 for combined visualization of multiple conditions. The top of the user interface 500 provides an overview of the critical conditions with multiple levels of resolution. A twenty four hour overview section 504, located on the left-hand side of the user interface 500, summarizes the patient's health status for the past 24 hours. A last hour overview section 505, located on the upper right-hand side of the user interface 500, summarizes the patient's health status for the past 60 minutes. A segment overview section 506, located just below the last hour overview section 505, summarizes the patient's health status for a segment of the last hour overview section 505.

The 3D anatomical condition visualization section 502, located on the left hand side of the user interface 500, includes a 3D visualization of a template body. The template body supports information visualization of multiple physiological conditions of the patient. The largest part of the interface 500 is allocated for the time series plot section 501, which provides the most detailed information, and can include: heart rate, respiratory rate, oxygen saturation of blood, systolic, diastolic or mean blood pressure. The system enables several synchronized channels (signals) to be displayed together in the time series plot section, so that dependencies between channels, or simultaneous changes of several channels can be identified. Each channel represents a time series signal generated from a corresponding physiological signal.

Scenarios for users interacting with the monitoring system depend on the amount of time the user can spend working with the system. For instance, a doctor starting his shift may want to know how the patient's condition has changed in the past 24 hours. The 24 hour overview section 504 informs the doctor if it is necessary to examine the data for the last 24 hours. Referring to FIG. 5b, a Search Event option 507 enables the doctor to effectively review alarms which are stored within the monitoring system's database. Alarms that have been activated for a particular physiological signal are presented in a table to enable the doctor to browse through them. If the doctor selects an alarm, the selected alarm is presented in the central window, together with the related data about the state of the patient when the alarm occurred and an automated written annotation. The automated written annotation is a description of why the system classified the event as critical. When there is only a single alarm, it is presented directly. Users who do not have much time to interact with the system can quickly determine whether a patient's condition is critical, or is likely to enter into a critical state.

The monitoring system enables users to quickly ascertain the conditions a patient is experiencing, or is likely to experience without examining the time series data or reading textual labels. In addition, an audible alarm is generated when an exceedingly critical condition occurs to alert users of the system.

Each interaction with a user may be logged. Users may also annotate critical events, which are then stored with the physiological signals in the database. The system may automatically generate a structured report, which may be included with the patient's record.

Although the exemplary embodiments of the present invention have been described in detail with reference to the accompanying drawings for the purpose of illustration, it is to be understood that the that the inventive processes and systems are not to be construed as limited thereby. It will be readily apparent to those of ordinary skill in the art that various modifications to the foregoing exemplary embodiments can be made therein without departing from the scope of the invention as defined by the appended claims, with equivalents of the claims to be included therein.

What is claimed is:

1. A method for anatomical visualization of a physiological signal, comprising the steps of:
acquiring a time-series signal from the physiological signal;
identifying a patient condition from the time-series signal;
displaying a 3D image of a body; and
displaying a visual indicator representative of the patient condition on the 3D image of a body.

2. The method of claim 1, wherein identifying the patient condition from the time-series signal comprises:
periodically extracting statistics of the time-series signal over a predetermined time period, wherein the statistics include one of moving averages of amplitude, minimum amplitude values, maximum amplitude values and slope trend information;
comparing the statistics with a predetermined library of statistical models to determine a match, wherein each of the statistical models corresponds to a patient condition; and
when the match is determined, outputting the patient condition.

3. The method of claim 1, wherein the visual indicator appears as a 3D anatomical structure representative of the corresponding physiological signal.

4. The method of claim 3, wherein the 3D anatomical structure changes periodically in color and brightness to indicate that the patient condition is critical or is approaching a critical state.

5. The method of claim 3, wherein the 3D anatomical structure has a constant color to indicate that the patient condition is stable.

6. The method of claim 3, wherein the 3D anatomical structure comprises vessels when the physiological signal is a blood pressure physiological signal.

7. The method of claim 3, wherein the 3D anatomical structure comprises skin when the physiological signal is a blood oxygen saturation physiological signal.

8. The method of claim 3, wherein the 3D anatomical structure comprises a heart when the physiological signal is a heart rate physiological signal.

9. The method of claim 3, wherein the 3D anatomical structure comprises a lung when the physiological signal is for a respiratory rate physiological signal.

10. The method of claim 1, wherein when the patient condition is exceedingly critical, an audible alarm is sounded.

11. The method of claim 1, wherein the 3D image of a body is derived from computer tomography data.

12. A method for visualizing a plurality of physiological signals, comprising the steps of:
acquiring a plurality of time-series signals from the corresponding plurality of physiological signals;
identifying a plurality of patient conditions from the time-series signals;
displaying a 3D image of a body; and
displaying a plurality of visual indicators on the 3D image of a body which correspond to the patient conditions.

13. The method of claim 12, wherein identifying the plurality of patient conditions from the time-series signals comprises:
periodically extracting statistics for a corresponding one of the time-series signals over a predetermined time period, wherein the statistics include one of moving averages of amplitude, minimum amplitude values, maximum amplitude values and slope trend information;
comparing the statistics with a predetermined library of statistical models to determine a match, wherein each of the statistical models corresponds to a patient condition; and
when the match is determined, outputting the patient condition.

14. The method of claim 12, wherein each of the visual indicators appears as an anatomical structure representative of the corresponding physiological signal.

15. The method of claim 14, wherein the anatomical structure changes periodically in color and brightness to indicate a critical condition or an approaching critical condition.

16. The method of claim 14, wherein the anatomical structure has a constant color to indicate a stable condition.

17. A computer readable medium having program instructions stored thereto for implementing the method claimed in claim 12 when executed in a digital processing device.

18. An apparatus for visualization of a plurality of physiological signals, comprising:
a time-series signal generation unit for acquiring a plurality of physiological signals and generating a plurality of time-series signals;
a patient condition analyzer unit for analyzing the time-series signals and generating patient condition data; and
a display unit for displaying the patient condition data as a 3D anatomical structure on a 3D template body.

19. The apparatus of claim 18, wherein the 3D anatomical structure is representative of a corresponding one of the physiological signals.

20. The apparatus of claim 18, wherein the 3D anatomical structure changes periodically in color and brightness when the patient condition data indicates a critical patient condition.

21. The apparatus of claim 18, wherein the anatomical structure remains a constant color when the patent condition data indicates a stable patient condition.

22. The apparatus of claim 18, further comprising an alarm unit for sounding an audible alarm when the patient condition data indicates an exceedingly critical patient condition.
